(19) United States
(12) Patent Application Publication
(10) Pub. No.: US 2022/0287606 A1
(43) Pub. Date: Sep. 15, 2022

(54) DISPOSABLE BLOOD METERING DEVICE

(71) Applicant: BD KIESTRA B.V., Drachten (NL)

(72) Inventors: Franciscus Feijen, Leeuwarden (NL); Johannes Anne Bruinsma, Drachten (NL); Samer Ahmed, Assen (NL); Roland Renkema, Drachten (NL); Daniel James Robertson, Leeuwarden (NL)

(73) Assignee: BD KIESTRA B.V., Drachten (NL)

(21) Appl. No.: 17/632,381

(22) PCT Filed: Aug. 5, 2020
(86) PCT No.: PCT/EP2020/072007

§ 371 Date: Feb. 2, 2022

Related U.S. Application Data
(60) Provisional application No. 62/883,294, filed on Aug. 6, 2019.

(51) Int. Cl. A61B 5/15; A61B 5/154

(52) U.S. Cl. CPC A61B 5/150946; A61B 5/15003; A61B 5/150221; A61B 5/154; A61B 5/15074

(57) ABSTRACT

Measurement system which can be used at patients bedside to monitor the amount of blood drawn from the patient. The system uses disposable sensor and electronics to measure accurately and in real time the volume of the blood drawn from the patient using a paddlewheel sensor wherein the rotation of the paddle wheel is correlated with the volume of the blood that is drawn from the patient and collected.

### RELATED APPLICATIONS

This application claims the benefit of U.S. Provisional Application No. 62/883,294, which was filed on Aug. 6, 2019 and is incorporated by reference herein.

### TECHNICAL FIELD

The apparatus described herein is a measurement system that can be used at a patient's bedside to monitor the amount of blood drawn from the patient. The system uses disposable actuation and sensor electronics to measure and control the amount of blood drawn from the patient for analysis.

### BACKGROUND

During blood collection for blood cultures from patients in hospital or other settings, it is important to provide the blood culture bottles with a targeted amount of blood to ensure that the drawn volume is neither too large nor too small, since inoculating the blood culture with an undersized and oversized sample can adversely affect the accuracy of the results of the blood culture analysis. At this moment the only feedback to the medical personnel (typically) drawing blood from a patient is visually monitoring the fluid level in the blood culture bottle during blood draw and discontinuing collection when the fill volume is determined to have been reached.

Currently the medical personnel make this determination visually. The blood culture bottle has a scale of volume measures on the bottle or the bottle label. Often, the medical personnel are required to mark the target filling volume for the blood on the side of the bottle. In practice, this method is susceptible to error. When a medical professional is drawing blood into the blood culture bottle, the medical personnel may not hold the bottle in a precisely vertical orientation, making it difficult or even impossible to determine the actual volume of the blood collected and making it likely that the target volume of the blood is not obtained. Another issue that can affect the accuracy of the volume of blood drawn is the lack of uniform instructions for how to properly inoculate the blood culture bottle with the target amount of blood. Also, the needs of the patient (who may have difficulties during the blood draw that might distract the medical personnel from accurately monitoring the blood draw) might adversely affect the accuracy of the volume of blood drawn by the medical personnel.

Successfully culturing and detecting a bacteria that has infected a patient is highly dependent on collecting the bacteria in the blood sample taken from the patient. The probability of having bacteria in the blood sample increases with an increase in the volume of blood collected. Therefore, collecting the target volume called for in a blood culture bottle, one example of which is a BACTEC™ culture bottle, with precision, is very important.

As noted above, currently, the medical personnel collecting the blood sample must visually determine when the correct volume of blood has been drawn and collected in the culture bottle, and stop the collection precisely at that point to avoid over-filling the blood culture bottle. Therefore, methods and apparatus for collecting blood that can ensure a target volume of blood is accurately collected continue to be sought.

### BRIEF SUMMARY

The blood metering device described herein measures the volume of blood that passes through it and flows into the blood collection vessel into which the device is attached. The blood collection vessel is any suitable container for receiving a blood samples. One example is a blood collection tube such a BD Vacutainer® tube. BD Vacutainer is a registered trademark of Becton, Dickinson and Company. Another example is a blood culture bottle such as the BACTEC bottle described above. The blood metering device provides at least one of: 1) an indication when a target volume of blood has passed through the device and into the blood culture bottle; or 2) an automatic shut off when a target volume of blood has passed through the device and into the blood culture bottle.

The blood metering device is a standard blood collection set in fluid communication with a mechanically rotating paddle wheel that rotates in response to the flow of blood through a housing in which the paddle wheel is rotatably mounted. The paddle wheel is positioned in the housing such that it rotates freely. In one embodiment the axis of rotation for the paddle wheel is a pin that is secured in the housing and defines the axis of rotation for the paddle wheel. The paddle wheel is in communication with a measuring sensor that can keep track of the rotations of the paddle wheel. One example of such a sensor is a small magnet that rotates with the paddle wheel and a hall effect sensor that is actuated as the magnet passes by the sensor. Each actuation is a rotation count. The sensor converts the number of rotations to blood volume. In some embodiments, the speed of the paddle wheel rotation is also measured to calculate the volume of the sample that passes through the blood metering device. Another example of a sensor is an optical sensor (e.g. a LED) that, in cooperation with an optical fiducial disposed on the paddle wheel, can count the number of rotations of the paddle wheel or the speed at which the paddle wheel is rotated, or both.

The blood metering device has a controller that can perform one or more of the following functions: i) associate the number of rotations with the volume of blood flowing through the device; ii) associate the speed at which the paddle wheel rotates with the volume of blood that passes through the paddle wheel; iii) turn off the blood flow in response to a determination that the target amount of blood has reached the target volume; iv) provide signals to the medical personnel regarding the volume of blood that has passed through the blood metering device. For example, the blood metering device could emit green light when the blood volume is below a certain threshold. As the blood volume that has passed through the device approaches the target volume, the green color might change to a yellow color. Once the target amount of blood volume has passed through the blood metering device and into the blood culture bottle, the sensor might change to yet another color (e.g. red) to indicate that the target volume has been received by the blood culture bottle. The blood does not flow through the sensor. In this regard the blood metering device is an assembly of a sensor unit and a metering/culture bottle adapter unit.

In one example, the sensor is disposable. In this example the sensor has disposable electronics that measure the amount of blood flowing through the blood metering device during blood draw from the patient. The disposable system informs the user if a pre-determined desired volume of blood has passed the sensor by means of a visual or acoustic signal.

The disposable sensor device is equipped with a sensor that can electronically measure blood flow. The disposable sensor unit is integrated in a disposable housing as part of the total blood collection set. The disposable sensor unit is removably attached to the culture bottle adapter unit, which contains the paddle wheel disposed in a housing and which is adapted to form a blood pathway from the blood collection system into the collection vessel (e.g. blood collection tube, blood culture bottle, etc.).

In some embodiments the sensor is not required to be disposable. In such embodiments the sensor unit does not come into contact with the blood, and therefore the sensor unit could be reused or recycled.

The blood filling volume is measured and monitored by a microprocessor which counts revolutions of the paddlewheel, or the rotation speed of the paddlewheel by means of a sensor, which is a calibrated and accurate measurement system. The system interacts with the user by means of optical and/or acoustical signals and/or other sensory signals (e.g. vibrations) to indicate that the predetermined volume of blood has been delivered into the blood culture bottle or blood collection vessel.

Optionally the blood metering device has an adapter unit that is a housing that defines a blood flow pathway and that is adapted to be connected to a blood collection set. The adapter unit has disposed therein a volume indicator that measures a volume of blood flowing through the blood flow pathway. Optionally that volume indicator is a paddle wheel flow detector. The volume indicator can also be a hair sensor, an acoustic sensor, or an optical sensor. The sensor can also be one of an axial rotor sensor, a peristaltic pump sensor, a magnetic field sensor, or rotating sensors.

In such a detector, the volume of blood that flows through the sensor is calculated from the number of rotations of the paddle wheel. The blood metering device also has a sensor unit that is engaged with the adapter unit. The sensor unit has: i) a sensor that is configured to detect signals from the sensor in response to blood flowing through the blood flow pathway in the adapter unit; and ii) a processor that associates the sensor signals with a blood volume and controls the response of the sensor unit in response to a determination by the sensor unit that a predetermined volume of blood has passed through the adapter unit. The sensor unit is one of detachably engaged with the adapter unit or monolithically integrated with the adapter unit.

The paddle wheel is disposed in the blood flow pathway but freely rotatable within the housing, for example by being supported on a pin in the housing that provides an axis of rotation. The paddle wheel has an axis of rotation and the axis of rotation is either orthogonal to a blood flow direction in the blood flow pathway or in line with a blood flow direction in the blood flow pathway. The paddle wheel can carry a magnet and the housing can have a hall effect sensor disposed thereon that is actuated as the magnet passes by the hall effect sensor.

12. The blood metering device of claim 11 wherein the paddle wheel rotates freely in the housing on an integrated pin supported by the housing.

Optionally the processor associates the rotation of the paddle wheel with a blood volume to determine a measured blood volume that has flowed through the blood metering device and controls the response of the sensor unit in response to the determination by the sensor unit that a predetermined volume blood has passed through the paddle wheel disposed in the adapter unit.

The adaptor unit is attachable to a collection vessel. The collection vessel can be a blood culture bottle or a sample collection tube.

Optionally, the processor compares the measured blood volume with the predetermined volume of blood and, when the measured blood volume is equal to the predetermined volume, the processor is configured to send a signal to close a blood flow valve that shuts off the flow of blood to the blood metering device.

Optionally, the adaptor unit has an activation lever that activates the processor when the adapter unit is attached to a blood culture bottle. The sensor unit optionally has a battery and the battery can be turned on by the activation lever, to power the processor.

The sensor unit optionally has a valve actuator that controls a valve in the adaptor unit. The valve actuator can be one of a moving magnet actuator, a micro actuator, a solenoid, or a paired magnet actuator.

The blood metering device optionally has a flowmeter that functions as a pump. One example of such a pump has a motor that has a rotor. The housing forms a stator for the pump. The rotor can have one or more magnets. The motor can have a hall effect sensor that measures a speed of rotation of the rotor. The processor determines the volume of blood flowing through the pump based on the speed of rotation of the motor. In operation, when the speed of rotation of the motor falls below a predetermined speed of rotation, the processor indicates a vein collapse. The sensor unit can have an indicator light that indicates that a predetermined volume of blood has passed through the adapter unit or a light the indicates a vein collapse based on signal from the processor.

The blood metering device is used by connecting the adaptor unit to a blood collection set with a needle adapted for venipuncture and tubing. In operation, when the speed of rotation of the motor falls below a predetermined speed of rotation, the processor indicates a vein collapse.

Also described herein is a method for determining a volume of blood flowing from a patient to a collection bottle. In the method an assembly of an adapter unit and a sensor unit is provided, the adapter unit has a housing that defines a blood flow pathway that is adapted to be connected to a blood collection set. Optionally, the adapter unit has disposed therein is a paddle wheel that is disposed in the blood flow pathway but freely rotatable within the housing. The sensor unit is as described above and has a sensor that is configured to detect signals from the sensor in response to blood flowing through the blood flow pathway in the adapter unit. The sensor unit also has a processor that associates the sensor signals with a blood volume and controls the response of the sensor unit in response to the determination by the sensor unit that a predetermined volume of blood has passed through the adapter unit. The sensor unit also has a valve actuator that is in signal communication with and is controlled by the processor. In the method, the assembly is connected to a blood collection set, the blood collection set having a needle adapted for venipuncture and tubing such that the blood collection set is in fluid communication with the blood flow pathway. The Adapter unit is connected to a blood collection vessel such that the blood flow pathway in the adapter is in fluid communication with the blood collection vessel. The pressure in the blood collection vessel is typically less than atmospheric pressure to draw the blood sample from the patient and into the blood collection vessel. This causes the blood to flow through a paddle wheel sensor and the rotation of the paddle wheel sensor is measured to determine the volume of blood flowing into the blood collection vessel from the blood flow pathway. The determined volume of blood is compared to the predetermined volume of blood. When the measured volume of blood equals the predetermined volume of blood, the processor sends a signal to the valve actuator to stop the blood from flowing into the collection vessel.

### BRIEF DESCRIPTION OF DRAWINGS

FIG. 1 illustrates an assembly for blood collection with a flow meter device coupled to a blood culture bottle;

FIG. 2A illustrates the flow meter electronics portion of the flow meter assembly;

FIG. 2B illustrates the flow meter adaptor portion of the flow meter assembly that attaches to the blood collection bottle;

FIG. 2C illustrates the blood flow path through the flow meter adaptor portion illustrated in FIG. 2B;

FIG. 3A illustrates a workflow using the flow meter device described herein in combination with a blood culture bottle;

FIG. 3B illustrates a workflow using the flow meter device describe herein in combination with a blood collection tube;

FIG. 4 is an exploded view of the flow meter adaptor portion illustrated in FIG. 2B;

FIG. 5 is an exploded view of the flow meter electronics portion illustrated in FIG. 2A;

FIG. 6 is a schematic of a flow meter device that illustrates theory of operation;

FIG. 7 illustrates the paddle wheel component of the flow meter adaptor portion;

FIG. 8 illustrates the housing the receives the paddle wheel coupled to a motor that drives the paddle wheel;

FIG. 9. illustrates one embodiment of a motor for driving the paddle wheel;

FIG. 10 illustrates an alternative assembly of the blood metering device and the blood culture bottle;

FIG. 11 is an exploded view of the assembly in FIG. 10;

FIG. 12 is the assembly of FIG. 10 integrated into a blood collection system;

FIG. 13 is a perspective phantom view of the blood metering device from the front of the disposable sensor unit integrated with the adaptor unit;

FIG. 14 is a perspective phantom view of the blood metering device from the rear of the sensor unit; and

FIG. 15 illustrates a pinch valve embodiment for use in the blood metering assembly of FIG. 1.

### DETAILED DESCRIPTION

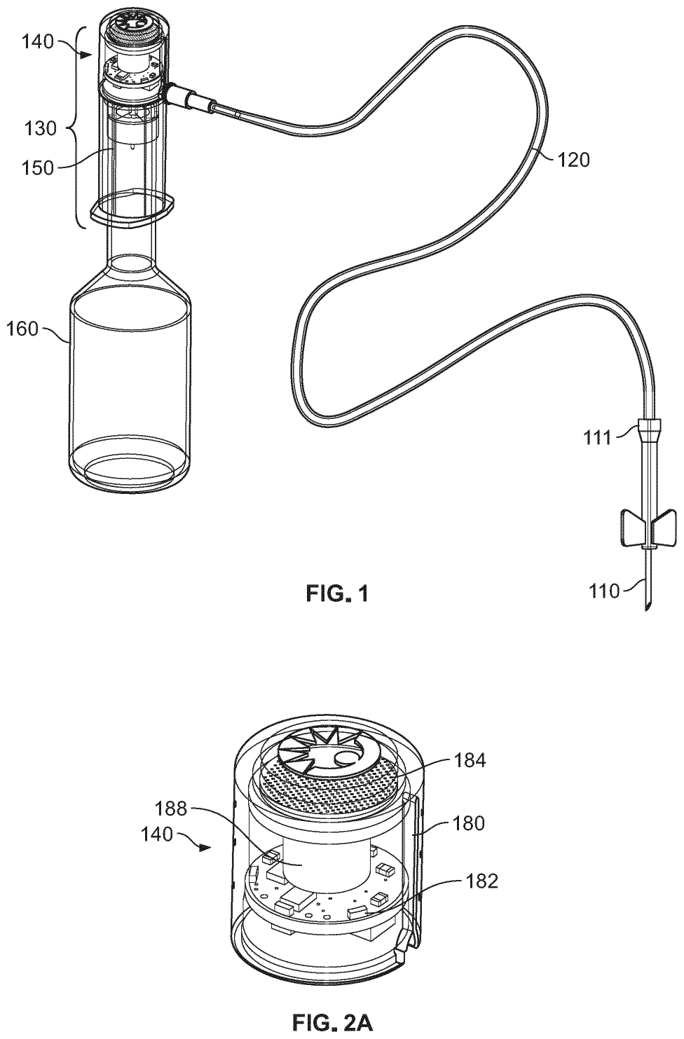

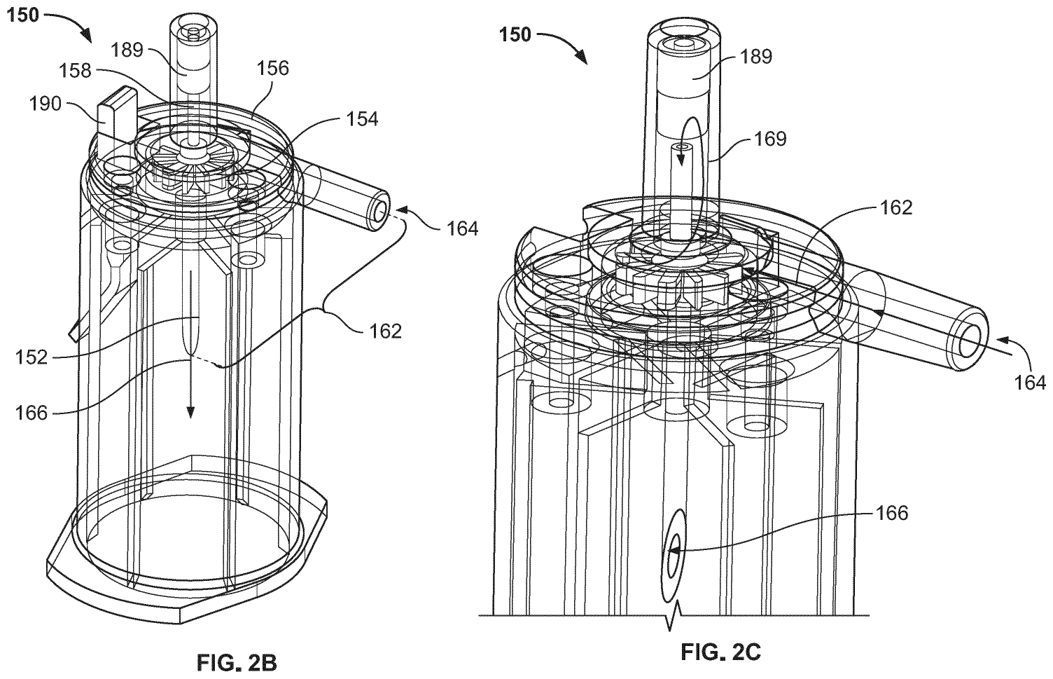

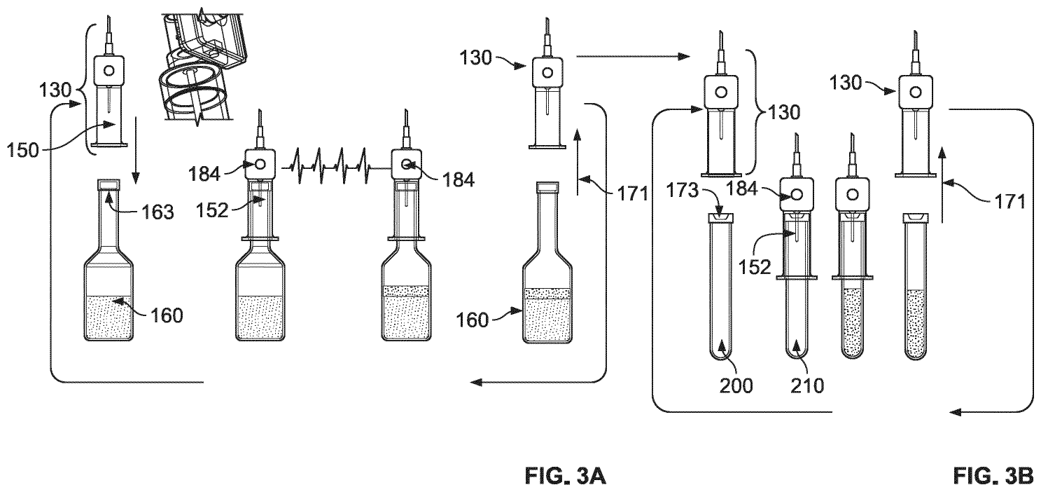

FIG. 1 illustrates a blood collection system comprising one embodiment of a blood metering device in accordance with the present technology. As shown in FIG. 1, the blood collection system includes needle 110, tubing 120, blood metering device 130, a sensor unit 140, adapter unit 150, and collection bottle 160. Adapter unit 150 includes needle 152 (FIG. 2B). Collection bottle 160 includes cap 163 (FIG. 3A). Needle 152 pierces through cap 163.

During the process of collecting a blood sample from a patient, needle 110 is used to pierce a vein or an artery of the patient. Driven by the vacuum pressure created by collection bottle 160, blood from the patient is directed toward collection bottle 160 through tubing 120. A flow of blood is collected in collection bottle 160. Along the way, the blood passes through the adapter unit 150 and needle 152. The sensor unit is also referred to as the electronics portion herein as the sensor unit contains the device actuator and the sensor electronics.

Referring to FIG. 2A, the sensor unit 140 has a housing 180 in which is disposed a processor 182, an indicator 184, a battery 186 and a valve actuator 188 that controls valve 189 on the housing inlet 164. The sensor unit contains the device actuator and the sensor electronics. The printed circuit board 182 (which carries the processor and other electronics), in response to the blood volume sensed by the metering device, can indicate when the predetermined target volume has passed through the metering device with an indicator (illustrated as colored light), but indication by audible signals or vibrations is also contemplated. The sensor can also send signals to other indicators of system conditions, such as an indication of other flow conditions (i.e. a blood flow rate that is higher or lower than a flow rate specified by the system).

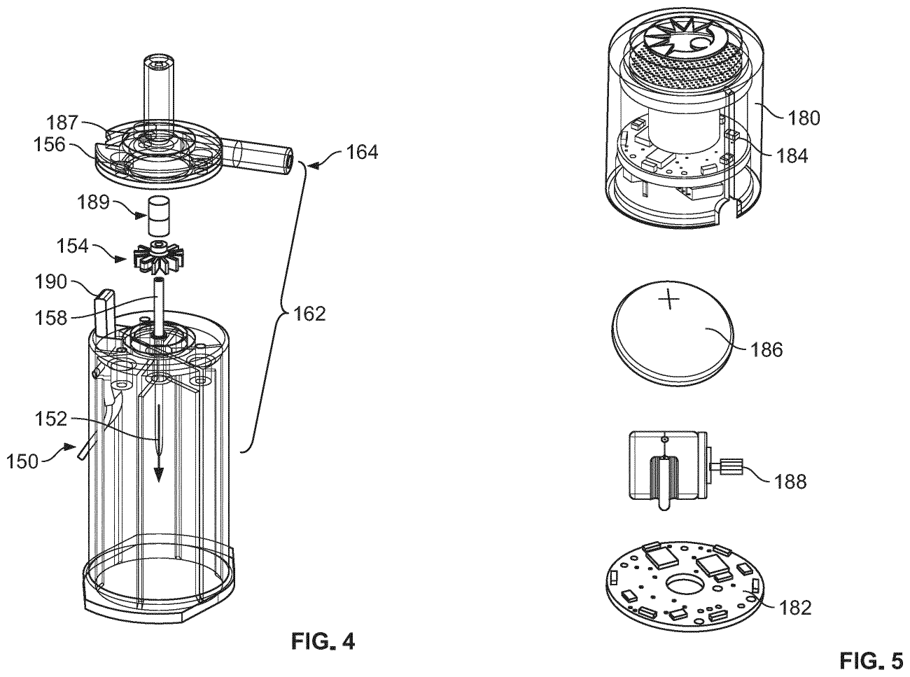

In one embodiment, the valve actuator 188 controls the flow of blood collected from the patient by keeping the valve 189 (FIG. 2B and FIG. 4) closed when blood draw from the patient commences. After blood draw is commenced, the valve actuator 188 receives a signal indicating that blood flow has started. In response to such signal, the valve actuator 188 gradually causes valve 189 to open. The valve actuator 188 is programmed to open the valve 189 in a manner that mitigates hemolysis of the blood flowing through the adaptor unit (FIG. 2B). In the illustrated embodiment the valve 189 is integrated with the adapter unit 150 illustrated in FIG. 2B. However, the valve 189 can also be integrated with the valve actuator 188. In either embodiment, the valve 189 is positioned in line with the inlet 164 in the adapter unit described below.

In an alternative embodiment, the sensor unit can be coupled (via wired or wireless communication) with a sensor 111 positioned near the needle 110. Should such sensor 111 detect flow conditions indicative of vein collapse or imminent vein collapse (i.e. a reduction in blood flow above predetermined threshold) the valve actuator 188 response is to shut the valve 189 followed by gradual reopening of the valve 189.

Suitable valve actuators are well known to one skilled in the art and are not described in detail herein. Such actuators include moving magnet actuators, micro actuators, solenoids, paired magnets, etc. that, in response to a signal, cause the valve 189 to open or close.

Suitable valves for use in the blood metering device disclosed herein are not described in detail herein and are well known to one skilled in the art. Examples of suitable valves include a shut off valve that advances a valve seat into a passage to turn off the valve and withdraws the valve seat from the passage to open the valve. Another suitable valve is a pinch tube valve 500. Such a valve is illustrated in FIG. 15. The pinch tube valve 500 is opened and closed by a solenoid 510 that drives a valve body 520 between an open and a closed position (and vice-versa). As illustrated in FIG. 15, tubing 530 passes through the valve body 520. Blood flows through the tubing 530 when the valve body 520 is opened. When in the open position, the valve body 520 does not pinch the tubing 530. When in the closed position, the valve body 520 closes on tube 530, preventing blood from flowing through the valve body 520. The solenoid 510 receives power through leads 540 positioned in solenoid cap 570. The pinch tube valve 500 also has a panel 550 and seal 560 to keep the solenoid from contact with the blood. The pinch tube valve 500 is provided with a manual override button 580 should the valve body malfunction and not release properly. Other suitable valves include ball valves, membrane valves, slide valves, check valves, release valves, etc.

Referring to FIG. 2B, the adaptor unit 150 has a small paddlewheel 154 which can rotate freely in a housing 156 on an integrated pin 158 in the housing 156. In the embodiment illustrated in FIGS. 2B and 4, the integrated pin 158 is part of the flow path 162 through the housing 156. The flow path exits the adapter unit 150 through outlet 166. The blood flow is tangentially directed through inlet 164 along the paddle wheel 154. The paddle wheel has a clearance with the housing 156 walls, so that the paddle wheel can rotate freely; there is no need for a tight sealing fit. The adaptor unit has an activation lever 190 that activates the electronics only after the adapter unit 150 is placed on the blood culture bottle 160 (FIG. 3A). This allows the device to be “off” when the device is not being used, thereby conserving the battery. When the needle 152 of the adaptor unit 150 pierces the blood culture bottle, the reduced pressure inside the blood culture bottle draws the patient blood through the device and into the blood culture bottle. Optionally, the metering device is configured so that the blood flow is axial through the metering device instead of tangential.

The blood flow path 162 through the adapter unit 150 is illustrated in FIG. 2C. The blood enters the adapter unit 150 through inlet 164. The blood flow path travels through paddle wheel 154 and then upward through channel 169 in which valve 189 is disposed. If the valve 189 is opened, blood is permitted to flow into and through the adapter unit outlet channel 166.

Operation of the device is illustrated in FIGS. 3A and 3B. Referring to FIG. 3A, the blood metering device 130 is attached to the culture bottle 160 by placing the adaptor unit 150 on the neck of the culture bottle 160 such that needle 152 pierces the cap 163. During blood draw, the indicator light 184 is one color (e.g. red). Optionally, the indicator light 184 will flash. Optionally, the flashing frequency will correlate with the blood flow. When the target draw volume is detected or the predetermined blood draw duration has been reached, the indicator light 184 turns a second color (e.g., green). Optionally, the metering device sends a signal to a valve actuator 188 that will cause the valve actuator 188 to close the valve to close that will shut off the flow of blood from the patient. The blood metering device 130 is then detached from the culture bottle 160. In one embodiment, the adapter unit 150 is spring-loaded, wherein the spring is biased to force the adapter unit from engagement with the culture bottle 160. During operation, the metering device is forced into engagement with the culture bottle or other collection vessel either by the operator or by a collection apparatus. Upon completion of collection, the force holding the adaptor unit 150 into engagement with the collection vessel is released, and the spring-loaded biasing force 171 of the adapter unit forces the adapter unit 150 from engagement with the collection vessel.

FIG. 3B illustrates an alternate work flow where the blood metering device 130 is used to collect blood 210 into a blood collection tube 200 instead of a culture bottle 160. The operation is as described above with regard to FIG. 3A. The septum cap 173 on the blood collection tube 200 is slightly different than the cap 163 on the blood culture bottle, but in operation needle 152 pierces the septum of septum cap 173 as it does the septum of cap 153.

FIG. 4 is an exploded view of the adapter unit 150 of FIG. 2B. The adapter unit is itself an assembly of the paddle wheel housing 156 that contains the inlet 164 with the adapter 150. The valve 189 and paddle wheel 154 are disposed between the paddle wheel housing 156 and the adapter 150. The paddle wheel is rotatably placed on pin 158. The flow path 162 of the blood through the adapter 164 is through the paddle wheel housing and out the needle 152. The activation lever 190 is disposed on the housing and fits through notch 187 in the paddle wheel housing 156. This enables activation lever 190 to be activated by placement of the sensor unit housing 180 on the paddle wheel housing 156.

FIG. 5 is an exploded view of the sensor unit 140 illustrated in FIG. 2A. The housing 180 has disposed therein a processor 182, an indicator 184, a battery 186 and a valve actuator 188 that controls valve 189 disposed adjacent the paddle wheel housing 156 of the adapter unit 150. The sensor unit 140 contains the device actuator and the sensor electronics. The printed circuit board 182 (which carries the processor and other electronics), in response to the blood volume sensed by the metering device, can either indicate when the predetermined target volume has passed through the metering device with an indicator 184 (illustrated as colored light), but indication by audible signals or vibrations is also contemplated).

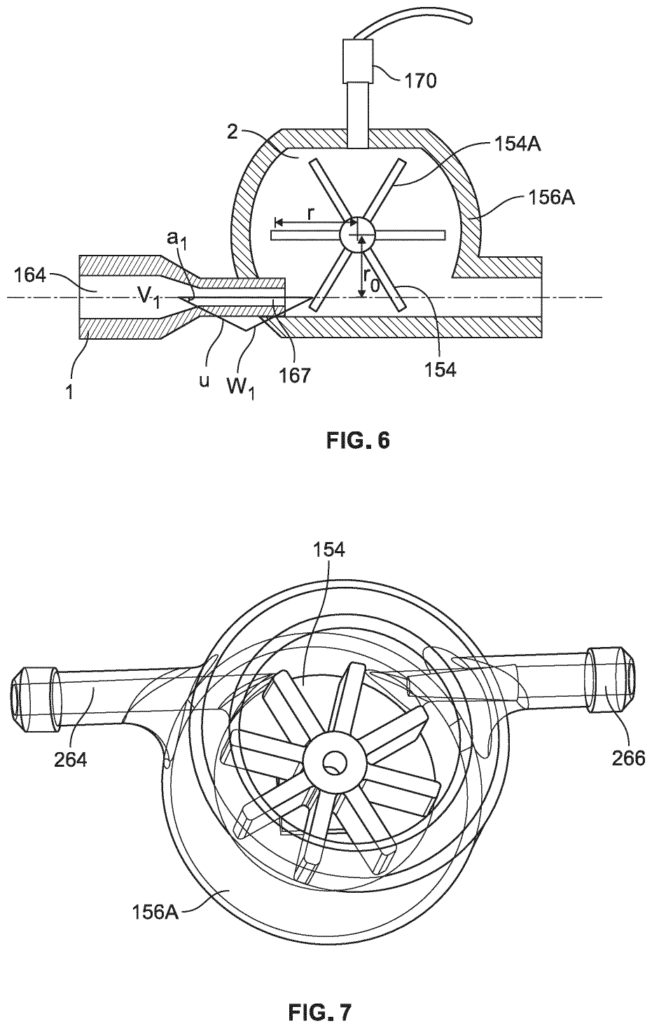

Referring to FIG. 6, the inlet 164 to housing 156 optionally has a small nozzle 167 which aims a concentrated jet of blood on the paddle wheel 154A. In the embodiment illustrated in FIG. 8, a small magnet 168 is integrated in the paddle wheel 154. A non-contact hall effect sensor (not shown but disposed in the sensor unit 140) can measure rotations of the magnet 168 through the housing 156A (in this housing the flow path 164 through the housing 156A is linear) in which the paddle wheel 154 is disposed. Other examples of sensors include an axial rotor sensor, in which a turbine is orthogonal to the blood flow direction. The turbine causes a rotor to rotate in response to the blood flowing through the turbine, and the rotation of the rotor is used to ascertain blood flow through the sensor. Other suitable sensors include peristaltic pump sensors, magnetic field sensors, and rotating sensors.

In one embodiment the blood metering device 130 is programmable to provide a few different selectable blood volume pre-sets of the blood volume passing through the paddle wheel 154. The pre-sets are the more common blood volumes (e.g. 10 mL) drawn from a patient.

Zhen, W., et al., “Computational study of the tangential type turbine flowmeter,” Flow Measurement and Instrumentation, Vol. 19, pp. 233-239 (2008), which is incorporated by reference herein, describes the calibration of a tangential type turbine flow meter. In FIG. 6, W₁ is the inlet velocity and rₒ is the axis between the shaft and the axis of the jet outlet. As described in Zhen et al., the rotor driving torque (Tᵣ) is calculated using the following equation:

Tᵣ=ρQ(V₁r cos α₁−V₂r cos α₂) (1)

where ρ is fluid density, Q is volumetric flow rate, r is the radius of the rotor, α₁ is the angle between V₁ and U₁ and α₂ is the angle between V₂ and U₂. The absolute velocity V₁ is determined by the equation:

V₁=Q/A (2)

where A is the jet aperture. The rotary speed (n) is calculated by:

V₂ cos α₂=u=2πrₒn (3)

From the above, the rotor driving torque is calculated. Meter performance is then calculated from the following equation:

Tᵣ−Tᵣₘ−T_{rf}−Tᵣₑ=0 (4)

where Tᵣ is the rotor driving torque, Tᵣₘ is journal bearing retarding torque, T_{rf} is rotor-blade retarding torque due to fluid drag and Tᵣₑ is retarding torque due to the attractive force of the magnetic pick-up. As further described in Zhen et al. these values are used to calculate a value for turbine meter performance. This enable volumetric flow rate to be determined from the rotor speed, the dimensions of the paddle wheel flow meter, etc.

The dimensions of the paddle wheel 154 and the housing 156 are largely a matter of design choice. A smaller dimensioned paddle wheel 154 will make more revolutions per mL of blood passing through the paddle wheel than a larger dimensioned paddle wheel. The width of the individual paddles 154A (FIG. 6) in the paddle wheel 156 should be slightly more than the width of the jet of blood (that width would be commensurate with the opening in the nozzle portion 167 if the housing inlet 164). Other contactless means of movement detection of the paddle wheel can be used, like a LED and photosensitive receiver. Such is illustrated as 170 in FIG. 6.

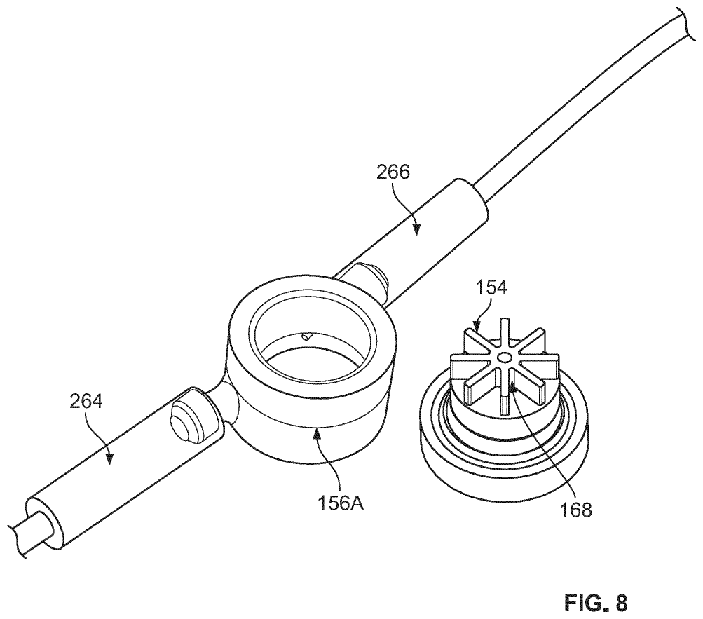

An alternative in-line housing 156A configuration is illustrated in FIGS. 7-8. In this configuration the housing inlet 264 and outlet 266 are in line and the blood flow path is linear. FIG. 2B illustrates the housing 156 with the housing inlet 164 orthogonal to the housing outlet 166.

The jet of blood is tangentially jetted on the paddle wheel 154, which causes a moment of force (or torque) on the paddle wheel 154 which, in turn, causes the paddle wheel 154 to turn. This is caused by the kinetic energy of the jet of blood. After first filling of the paddle wheel housing 156 with blood, air bubbles could form and obstruct the movement of the paddle wheel 154.

As described above, the relationship between the number of revolutions of the paddle wheel and the actual blood volume passed is not linear. Besides the driving jet of fluid on the paddle wheel there is also a dampening action of the paddle rotating in this fluid. This causes “slip”, which will vary due to differences in pressure and viscosity. Optionally, the behavior of the paddle wheel can be monitored and modeled to predict the slip based on flow conditions. Once the slip is determined, the flow conditions can be provided to the processor and the processor can factor in the slip to correct for the volume that is calculated based on the number of revolutions of the paddle wheel. This could lead to large fluctuations in the volume actually metered with the measured metered volume.

Optionally, the device will be calibrated to correlate the measured metered volume with the actual metered volume. This will ensure that the blood meter device described herein accurately draws the targeted blood volume (typically between 8 mL to 10 mL of blood) at all times. The speed at which the blood is drawn will also influence the accuracy of the volume measured. It is contemplated that the metering device described herein will be calibrated such that the effect of flow rate on measured volume is known. In one embodiment, the revolution of the paddle wheel is correlated with the volume of blood that flows through the paddle wheel. In an alternative embodiment, the speed of rotation (i.e. the RPM of the paddle wheel) is used to determine flow rate which in turn is used to calculate the volume of blood that is passing through the paddle wheel. Once calibrated, the metering device measures the speed of blood flow and adjusts the measured volume to compensate for known inaccuracies in volume measurement at certain blood flow rates. Optionally, the blood metering device has a switch to power up and reset the system every time a new blood culture bottle is presented for filling.

Although the embodiments herein describe a paddle wheel flow meter, other metering devices are contemplated such as a hair sensor, acoustic sensor, optical sensor, etc. Such sensors are well known to the skilled person and not described in detail herein. In some embodiments in which the sensor unit does not come into contact with the blood, the sensor unit could be reused.

As described previously, a combined flowmeter/pump the blood metering device described herein can be configured to detect a vein collapse (by detecting reduced or inadequate blood flow) and re-inflate veins (by stopping blood flow through the metering device but not removing the needle for blood draw from the patient). As stated above, the blood metering device described herein can be actuated when the device has determined that the target amount of blood has been drawn, thereby stopping the flow of blood through the metering device.

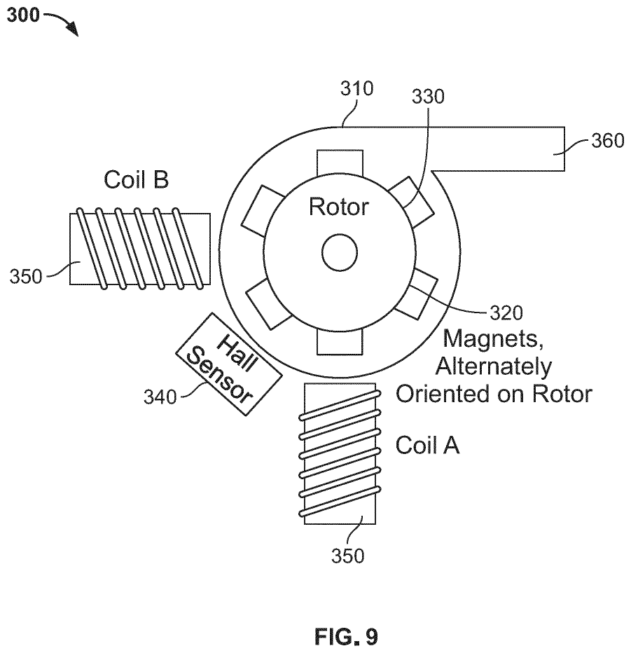

For actively metering the blood flow, a low intensity commutating magnetic field can be induced by the controller, to help the rotor turn at low flowrates. A disposable flow meter/pump 300 is illustrated in FIG. 9. As illustrated, the stator 310 (i.e. the housing) and rotor 320 are fully separated and can easily be taken apart. The rotor 320 can be a one piece sintered and magnetized part. Magnets 330 are placed on the rotor 320. A hall effect sensor 340 detects the passing magnets and determines the rotor RPM. From the resulting RPM, the volume of blood flowing into the culture bottle is determined. In the pump function, the rotor is driven by coils A and B 350. For the device illustrated in FIG. 9 it is noted that the flow meter and pump functions cannot be performed simultaneously. The hall sensor can be eliminated by measuring the back EMF from coils 350.

The magnets 330 on the rotor 320 also function as paddles (such as the paddles 154A in FIG. 6). Therefore, the motor 300 can either function as a paddle wheel flow sensor, or when driven by the coils 350, function as a centrifugal pump. The housing 310 around the rotor 320 is air/water tight, and made of a non-conductive material so it does not interfere with the magnetic fields needed to drive the rotor. There is a tangential inlet/outlet 360 into the housing, as well as an axial inlet/outlet (not illustrated in FIG. 9). Optionally, the two coils are positioned such that the coils and hall sensor cover less than 180 degrees of the circumference of the rotor. This makes easy disassembly very easy, when compared with current stator designs which cover full 360 degree.

The motor 300 is provided with a commutated low power rotating magnetic field to help drive the paddle wheel in the device even at low flowrates. The rotor 320 is optionally made of a single piece of magnetizable material. The rotor 320 is optionally ring-shaped with protrusions 330 on the outer circumference that act as magnetic poles as well as paddles. The stator 310 has at least 2 poles, which is why two coils, 350, are illustrated. The coils 350 are positioned no more than 180 degrees apart on the stator. This ensures easy assembly/disassembly of rotor/housing 320 and stator 310. The illustrated device 300 can be incorporated into a device that measures blood flow and/or pumps blood, medicine, sample, reagents, etc. either into a patient or into a vessel such as a collection tube. The poles/magnets are oriented radially in the example, they could also be oriented axially. The motor is preferably synchronous, but can also be operated using asynchronous commutation.

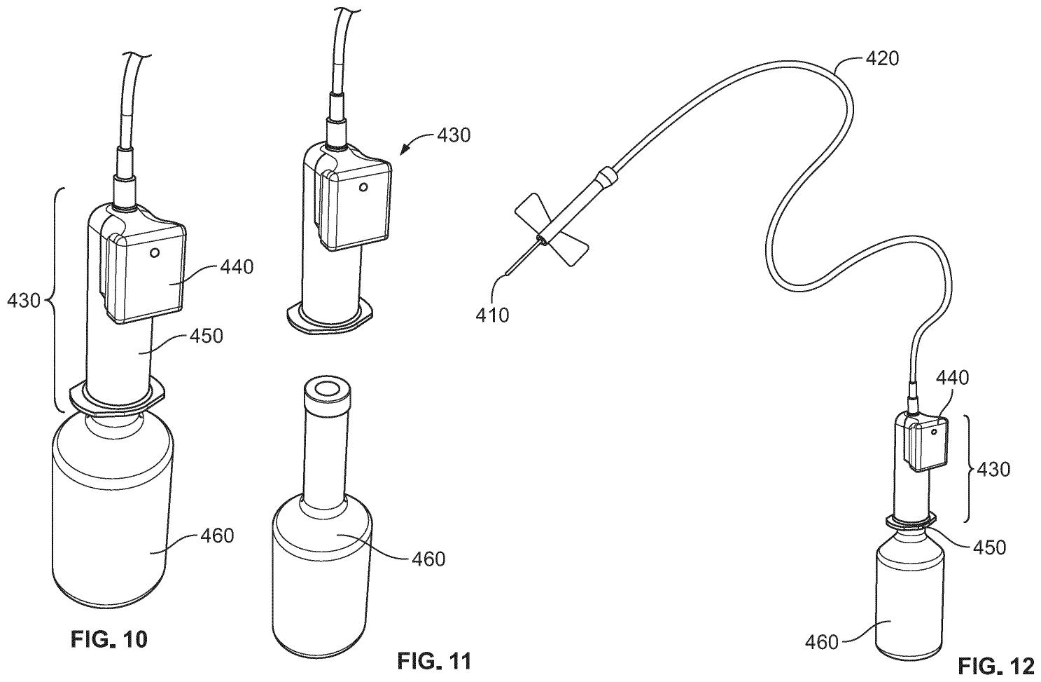

FIG. 10 illustrates an alternative configuration of the blood metering device and the blood culture bottle. The blood metering device 430 is attached to blood culture bottle 460. In this embodiment the sensor portion 440 is monolithically integrated with the adapter portion 450 to form the blood metering device 430.

FIG. 11 is an exploded view of the assembly in FIG. 10. In this illustration the monolithic blood metering device 430 is removed from the blood culture bottle 460.

FIG. 12 illustrates a blood collection system that includes needle 410, tubing 420, blood metering device 430, sensor portion 440, adapter portion 450, and collection bottle 460. During the process of collecting a blood sample from a patient, needle 410 is used to pierce a vein or an artery of the patient. Driven by the vacuum pressure created by collection bottle 460, blood from the patient is directed toward collection bottle 460 through tubing 420. The blood is collected in collection bottle 460.

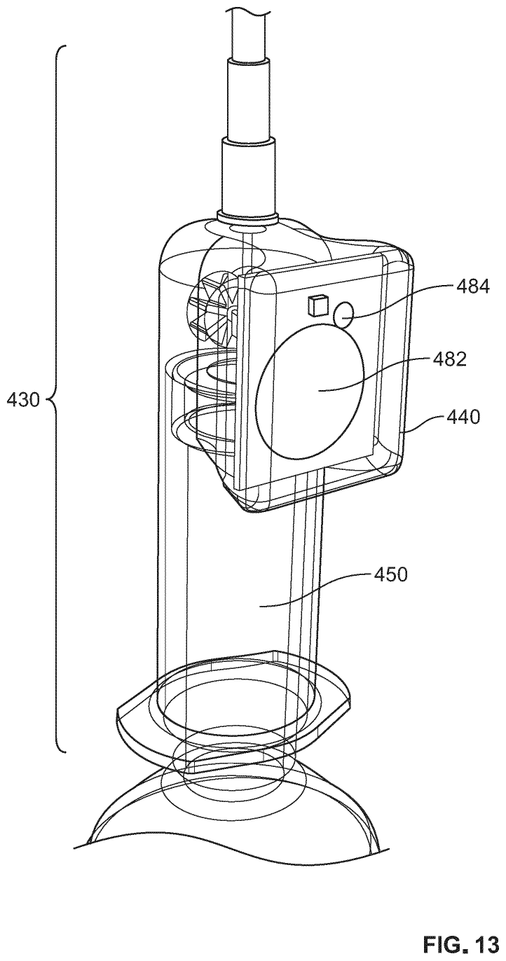

FIG. 13 is a perspective phantom view of the disposable blood metering device 430 from the front of the sensor portion 440 integrated with the adaptor portion 450. The blood metering device 430 has a processor 482 that has a small embedded memory and a disposable printed circuit board (PCB). The processor embedded memory has stored therein information that controls the operation of the blood metering device. Non-limiting examples of such information includes total blood volume that passes through the device (i.e. the predetermined fill volume); the maximum duration of the blood draw (after which time the device terminates further collection of the blood from the patient); and changes in blood flow rate from the patient indicative of vein collapse). The LED indicator 484 provides an indication of fluid (e.g. blood) volume that has passed through the blood metering device 430. Other indicators (both to the user and the actuator) that the predetermined fill volume has been received by the container include sensory alerts such as a vibration alert.

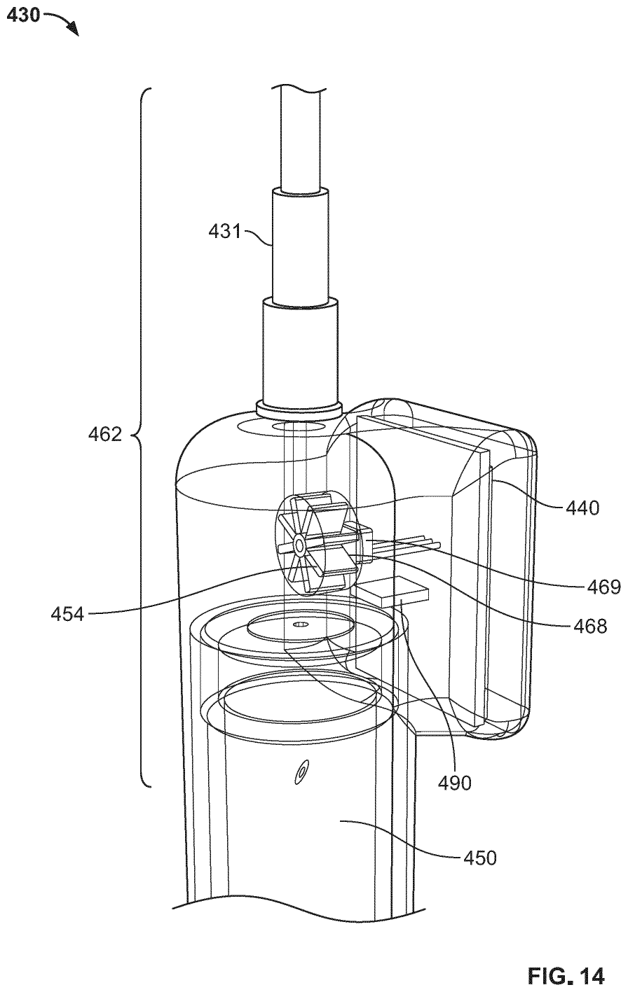

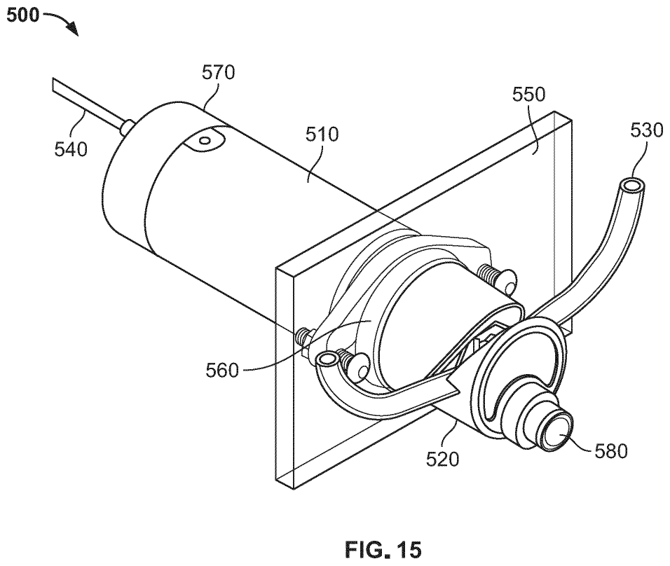

FIG. 14 is a perspective phantom view of the blood metering device 430 from the rear of the sensor portion 440 integrated with the adapter portion 450. The blood metering device 430 has the paddle wheel 454 placed in the blood flow pathway 462 that enters through the top 431 of the blood metering device 430. A hall sensor 469 senses the magnet 468 in the paddle wheel and the number of turns senses by the hall sensor 468 is converted into volume by the processor 482. The mechanical contact 490 senses contact of the blood metering device 430 with the collection bottle 460 and initiates blood draw from the patient into the collection bottle 460.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive. For example, whilst the disclosure has described the collection of blood in a blood culture bottle, the same principal is applicable to the collection of other fluids in other containers.

It will further be understood that any reference herein to subject matter known in the field does not, unless the contrary indication appears, constitute an admission that such subject matter is commonly known by those skilled in the art to which the present technology relates.

1. A blood metering device comprising:
an adapter unit comprising a housing that defines a blood flow pathway that is adapted to be connected to a blood collection set, wherein the adapter unit has disposed therein a volume indicator that measures a volume of blood flowing through the blood flow pathway;
a sensor unit that is engaged with the adapter unit; the sensor unit comprising: i) a sensor that is configured to detect signals from the sensor in response to blood flowing through the blood flow pathway in the adapter unit; and ii) a processor that associates the sensor signals with a blood volume and controls the response of the sensor unit in response to a determination by the sensor unit that a predetermined volume of blood has passed through the adapter unit.

2. The blood metering device of claim 1, wherein the sensor unit is one of detachably engaged with the adapter unit or monolithically integrated with the adapter unit.

3. The blood metering device of claim 1, wherein the volume indicator is a paddle wheel that is disposed in the blood flow pathway but freely rotatable within the housing.

4. The blood metering device of claim 3, wherein the paddle wheel has an axis of rotation and the axis of rotation is orthogonal to a blood flow direction in the blood flow pathway.

5. The blood metering device of claim 3, wherein the paddle wheel has an axis of rotation that is in line with a blood flow direction in the blood flow pathway.

6. The blood metering device of claim 3, wherein the processor associates the rotation of the paddle wheel with a blood volume to determine a measured blood volume that has flowed through the blood metering device and controls the response of the sensor unit in response to the determination by the sensor unit that a predetermined volume blood has passed through the paddle wheel disposed in the adapter unit.

7. The blood metering device of claim 1, wherein the adaptor unit is attachable to a collection vessel, wherein the collection vessel is selected from the group consisting of blood culture bottles and sample collection tubes.

8. The blood metering device of claim 6, wherein the processor compares the measured blood volume with the predetermined volume of blood and, when the measured blood volume is equal to the predetermined volume, the processor is configured to send a signal to close a blood flow valve that shuts off the flow of blood to the blood metering device.

9. The blood metering device of claim 1, wherein the volume indicator is one of a hair sensor, an acoustic sensor, and an optical sensor.

10. The blood metering device of claim 3, wherein the sensor is one of an axial rotor sensor, a peristaltic pump sensor, a magnetic field sensor, and rotating sensors.

11. The blood metering device of claim 3, wherein the paddle wheel carries a magnet and the housing has a hall effect sensor disposed thereon that is actuated as the magnet passes by the hall effect sensor.

12. The blood metering device of claim 11, wherein the paddle wheel rotates freely in the housing on an integrated pin supported by the housing.

13. The blood metering device of claim 1, wherein the housing defines a blood flow pathway wherein the flow path exits the adapter unit through an outlet.

14. The blood metering device of claim 1, wherein the adaptor unit comprises an activation lever that activates the processor when the adapter unit is attached to a blood culture bottle.

15. The blood metering device of claim 1, wherein the sensor unit comprises a battery.

16. The blood metering device of claim 1, wherein the sensor unit comprises a valve actuator.

17. The blood metering device of claim 16, wherein the valve actuator is one of a moving magnet actuator, a micro actuator, a solenoid, or a paired magnet actuator.

18. The blood metering device of claim 1, wherein the volume indicator is a combination of a flowmeter and a pump.

19. The blood metering device of claim 18, wherein the pump comprises a motor, the motor comprising a rotor and wherein the housing forms a stator for the pump.

20. The blood metering device of claim 19, wherein the rotor comprises one or more magnets.

21. The blood metering device of claim 20, further comprising a hall effect sensor that measures a speed of rotation of the rotor.

22. The blood metering device of claim 21, wherein the processor determines the volume of blood flowing through the pump based on the speed of rotation of the motor.

23. The blood metering device of claim 22, wherein the blood collection set comprises a needle adapted for venipuncture and tubing.

24. The blood metering device of claim 23, wherein, in operation, when the speed of rotation of the motor falls below a predetermined speed of rotation, the processor indicates a vein collapse.

25. The blood metering device of claim 1, wherein the sensor unit has an indicator light that indicates that a predetermined volume of blood has passed through the adapter unit based on signal from the processor.

26. The blood metering device of claim 24, wherein the sensor unit has an indicator light that indicates that a vein collapse has occurred based on a signal from the processor.

27. A blood metering device comprising:
an adapter unit comprising a housing that defines a blood flow pathway that is adapted to be connected to a blood collection set, wherein the adapter unit has disposed therein is a paddle wheel that is disposed in the blood flow pathway but freely rotatable within the housing;
a sensor unit that is engaged with the adapter unit; the sensor unit comprising:
i) a sensor that is configured to detect signals from the sensor in response to blood flowing through the blood flow pathway in the adapter unit;
ii) a processor that associates the sensor signals with a blood volume and controls the response of the sensor unit in response to a determination by the sensor unit that a predetermined volume of blood has passed through the adapter unit; and
iii) a valve actuator that is in signal communication with and is controlled by the processor.

28. The blood metering device of claim 27, wherein the adaptor unit comprises an activation lever that activates the processor when the adapter unit is attached to a blood culture bottle.

29. The blood metering device of claim 28, wherein the processor associates the rotation of the paddle wheel with a blood volume to determine a measured blood volume that has flowed through the blood metering device and controls the response of the sensor unit in response to the determination by the sensor unit that a predetermined volume blood has passed through the paddle wheel disposed in the adapter unit.

30. A method for determining a volume of blood flowing from a patient to a collection bottle the method comprising:
providing an assembly of an adapter unit and a sensor unit, the adapter unit comprising a housing that defines a blood flow pathway that is adapted to be connected to a blood collection set, wherein the adapter unit has disposed therein is a paddle wheel that is disposed in the blood flow pathway but freely rotatable within the housing;
wherein the sensor unit comprises:
i) a sensor that is configured to detect signals from the sensor in response to blood flowing through the blood flow pathway in the adapter unit;
ii) a processor that associates the sensor signals with a blood volume and controls the response of the sensor unit in response to the determination by the sensor unit that a predetermined volume of blood has passed through the adapter unit; and
iii) a valve actuator that is in signal communication with and is controlled by the processor;
connecting the assembly to the blood collection set, the blood collection set comprising a needle adapted for venipuncture and tubing such that the blood collection set is in fluid communication with the blood flow pathway;
connecting the adapter unit to a blood collection vessel such that the blood flow pathway in the adapter is in fluid communication with the blood collection vessel, wherein a pressure in the blood collection vessel is less than atmospheric pressure;
collecting a blood sample from a patient by venipuncture of the patient with the needle, thereby causing blood to flow through the blood flow pathway to the blood collection vessel;
flowing the blood through a paddle wheel sensor;
measuring the rotation of the paddle wheel sensor;
measuring the volume of blood flowing into the blood collection vessel from the blood flow pathway; and
comparing the determined volume of blood with a predetermined volume of blood;
